(19) United States
(12) Patent Application Publication
(10) Pub. No.: US 2025/0152005 A1
(43) Pub. Date: May 15, 2025

(54) OPHTHALMIC INSTRUMENT FOR SELF-TONOMETRY

(71) Applicant: Eye to Eye Telehealth, Inc., Cheyenne, WY (US)

(72) Inventors: David Smits, Cheyenne, WY (US); Preston Smits, Denver, CO (US)

(21) Appl. No.: 18/838,138

(22) PCT Filed: Feb. 15, 2023
(86) PCT No.: PCT/US2023/062620

§ 371 Date: Aug. 13, 2024

Related U.S. Application Data
(60) Provisional application No. 63/310,218, filed on Feb. 15, 2022.

(51) Int. Cl. A61B 3/16; A61B 3/00

(52) U.S. Cl. CPC A61B 3/16; A61B 3/0066; A61B 2562/0219; A61B 2562/0257

(57) ABSTRACT

An ophthalmic instrument for self-tonometry includes a body that houses a measurement device for measuring intraocular pressure of a user's eye and at least one anatomical fixation point for adjusting a distance between the anatomical fixation point and a center of the user's eye. An eye identification device attached to the body uses a gyroscope or other sensor to allow identification of a left eye or a right eye being measured.

### FIELD OF INVENTION

The present invention relates to an apparatus for self-measurement of intraocular pressure, particularly with regard to structure-function relationship between a user and their eye(s).

### BACKGROUND

Measuring intraocular pressure (IOP) is important for the assessment of eye health. Glaucoma is a chronic progressive blinding disease which is monitored by measuring intraocular pressure and treated by lowering intraocular pressure.

Intraocular pressure is measured with a device referred to as a tonometer. Goldman Applanation Tonometry (GAT) remains the reference-standard tonometry method and involves measuring the force required to flatten the cornea with a prism of known surface area. However, GAT has limitations to its use, requiring instillation of anesthetic drops, bulky and expensive equipment, and patient positioning difficulty. Additional methods of tonometry have been described, such as in U.S. Pat. No. 5,299,573 which uses a non-contact method wherein compressed air is sprayed on the cornea with subsequent measurement of tissue deformation.

Intraocular pressure measurement most commonly occurs in a clinic by a skilled healthcare provider. Measurements occur infrequently, often many months apart, at single and discrete points in time. However, in reality, intraocular pressure has diurnal fluctuation with troughs and peaks. Studies have shown more than 50-75% of IOP peaks occur outside clinic hours. Thus, a single IOP measurement by tonometry in a clinic does not accurately portray a patient's true IOP profile and subsequent risk of vision loss.

As such, there is a need for tonometry devices that can be used by the patient to check their own pressure at home to capture IOP data that would otherwise be missed from in-clinic measurement. The requirements of such a device are unique as the patient must self-align and stabilize a device for measurement. One such alignment mechanism has been described using patient visualization of light-channels in U.S. Pat. No. 10,058,245. However, alignment alone does not fulfill all the design input requirements for successful self-tonometry, which necessitates additional means for stabilization, and eye identification.

### SUMMARY

The present invention provides an ophthalmic instrument that satisfies the user's needs for successful home-tonometry measurement, in which alignment, stabilization, and eye identification are all accomplished by the apparatus.

The ophthalmic instrument includes a measurement device and one or more anatomical fixation points. The measurement device is housed in or on the body of the instrument and is used for measuring intraocular pressure (IOP) of the user's eye. The body is intended to be held and stabilized with two hands and rested on a part of the user's anatomy for alignment and subsequent measurement. In one embodiment, the body is rested on the user's nose, though it is conceivable that it may be rested on any other number of the user's anatomical facial structures, such as a cheek, forehead, brow, or any other orbit bones.

The anatomical fixation point is a point or area on the body of the instrument that rests against a part of the user's anatomy, such as the user's nose, cheek, forehead, or brow, and is used for aligning the user's eye to the measurement device. According to one embodiment, the anatomical fixation point is in the form of eye cups designed to rest on the user's orbit bones. With a structure similar to binoculars, the distance between the two eye cups can be adjusted to fit the user's pupillary distance. According to another embodiment, the anatomical fixation point has a horizontal length to accommodate a range of distances for aligning the user's eye to the measurement device, which can be adjusted by the user. More particularly, the distance adjusted by the user, between the anatomical fixation point and the center of the user's eye, may represent the pupillary distance. The instrument may include one or more pads on an outer surface of the body, specifically on the anatomical fixation point, to provide comfort when resting against a part of the user's anatomy.

An eye identification device is also attached to the body of the instrument and allows identification of a left eye or a right eye being measured. According to one embodiment, the eye identification device includes a gyroscope or other sensor that detects rotation of the body about a Z-axis 180 degrees in relation to an asymmetry of the body with respect to a Y-axis. According to another embodiment, the eye identification device includes one or more sensors that sense the user in relation to the measurement device. The sensors may be contact sensors or proximity sensors, for example. According to certain embodiments, the ophthalmic instrument may include both a gyroscope and one or more additional sensors.

### BRIEF DESCRIPTION OF THE DRAWINGS

FIG. 1 is a perspective view of an ophthalmic instrument for self-tonometry.

FIG. 2 is a top view of the ophthalmic instrument.

FIGS. 3 and 4 show the ophthalmic instrument in use.

FIG. 5 is a top view of another embodiment of the ophthalmic instrument for self-tonometry.

FIG. 6 shows the ophthalmic instrument in use.

FIG. 7 is a diagram of the ophthalmic instrument for self-tonometry.

FIGS. 8a and 8b are front views of the ophthalmic instrument for self-tonometry, illustrating rotation about a Z-axis.

FIG. 9 is a front view of another embodiment of the ophthalmic instrument for self-tonometry.

FIG. 10 is a front view of yet another embodiment of the ophthalmic instrument for self-tonometry.

### DETAILED DESCRIPTION

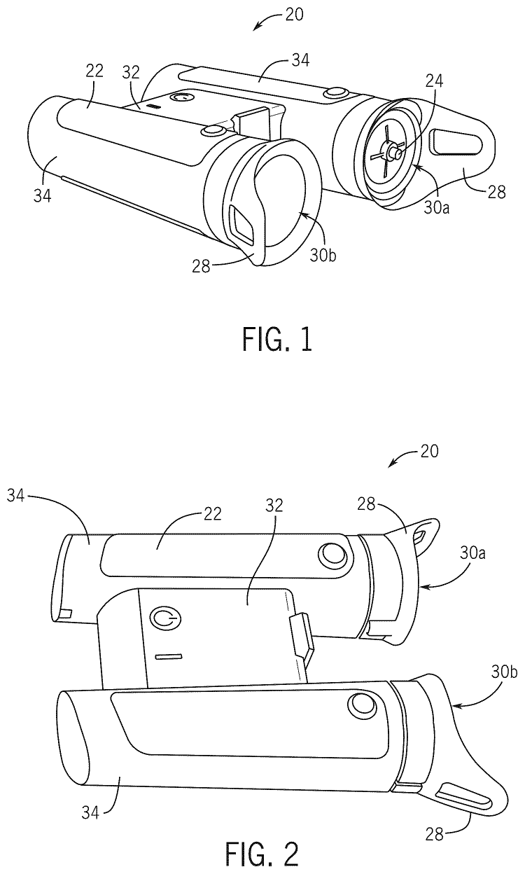

An ophthalmic instrument 20 for self-tonometry is illustrated in FIGS. 1-10. A user can use the ophthalmic instrument 20 by themselves in the comfort of their own home to monitor intraocular pressure (IOP) at any time. The ophthalmic instrument 20 provides ease of use and accurate measurements through proper alignment, stabilization, and eye identification.

The ophthalmic instrument 20 includes a body 22 that houses a measurement device 24 and one or more anatomical fixation points 28 located on the body 22 of the instrument 20, which rest against a part of the user's anatomy during use. The anatomical fixation points 28 enable a user to adjust a distance between the anatomical fixation point 28 and a center of the user's eye in which the IOP is being measured. The measurement device 24 is positioned inside of or on the body 22 of the instrument 20, and measures IOP of the user's eye. The instrument 20 may be used to measure IOP in either a right eye or a left eye. An example of a suitable measurement device 24 is a rebound tonometer or non-contact tonometer.

In the embodiment illustrated in FIGS. 1-4, the ophthalmic instrument 20 includes two anatomical fixation points 28 in the form of eye cups 30 designed to rest on the user's orbit bones. The measurement device 24 in this embodiment is positioned within the cavity of one of the eye cups 30a, while the other eye cup 30b is opaque or dark, essentially occluding the other eye's vision so that the user can focus on a measurement target. With a structure similar to binoculars, the distance between the two eye cups 30 can be adjusted along an X-axis to fit the user's pupillary distance, so that both eye cups 30 are aligned with the user's eyes, as visually determined by the user. More particularly, a central portion 32 of the body 22 can connect two tubular structures 34, each tubular structure 34 having an eye cup 30 at one end. The tubular structures 34 can be affixed to the central portion 32 in a hinged manner such that the tubular structures 34 can swivel about a Z-axis to allow the tubular structures 34 to move toward one another or away from another; additionally or alternatively, the tubular structures 34 can be affixed to the central portion 32 in a sliding configuration to allow the tubular structures 34 to move toward one another or away from another.

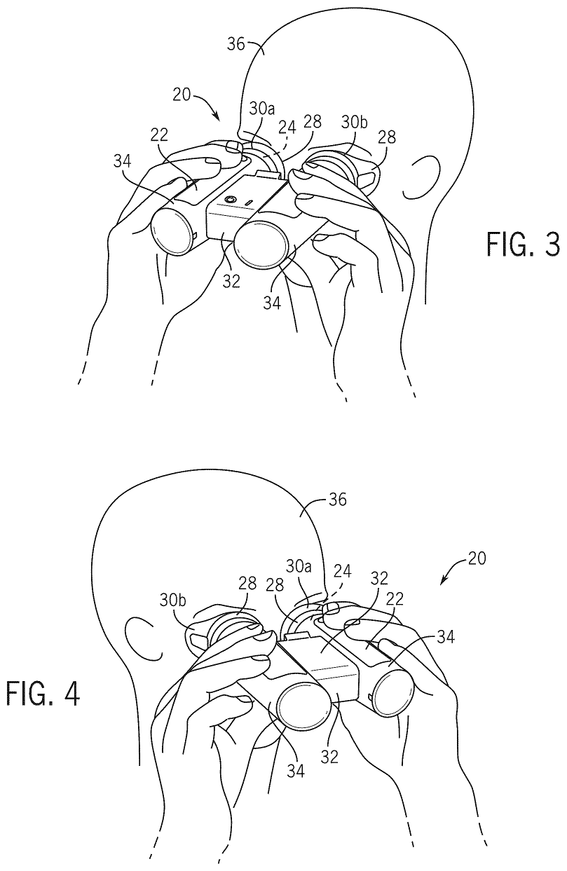

FIGS. 3 and 4 each show the ophthalmic instrument 20 in use, with a user 36 holding the body 22, stabilized through the use of two hands, and the two anatomical fixation points 28 are rested against the user's orbit bones for alignment and subsequent measurement. The embodiment having two eye cups 30 provides substantial stability of the ophthalmic instrument 20 in use. In fact, the two-eye-cup embodiment provides several additional advantages. For example, one of the eye cups 30a can include the measurement device 24 while the other eye cup 30b can include an opaque cover or other non-functional eye-facing surface. The non-functional eye-facing surface masks the contralateral eye, making it easier for the user 36 to focus on alignment crosshairs in the measurement eye without having to strain by closing one eyelid. It has been shown that closing one eyelid causes artificially high IOP in the measurement eye. Additionally, the inclusion of two eye cups 30 enables the user to provide perfect alignment with both gross- and fine-tuning of the measurement device 24 with the pupil. Furthermore, the inclusion of two eye cups 30 provides a feeling of symmetry and a visual style of binoculars, which makes it easier for a user 36 to understand how to use the ophthalmic instrument 20.

FIG. 4 illustrates the ophthalmic instrument 20 in an inverted position relative to FIG. 3. In FIG. 3, the user 36 is holding the ophthalmic instrument 20 with the first eye cup 30a that houses the measurement device 24 aligned with the user's right eye in order to measure IOP in the user's right eye. In FIG. 4, the user 36 has rotated the ophthalmic instrument 20 about a Z-axis and is holding the ophthalmic instrument 20 with the first eye cup 30a aligned with the user's left eye in order to measure IOP in the user's left eye.

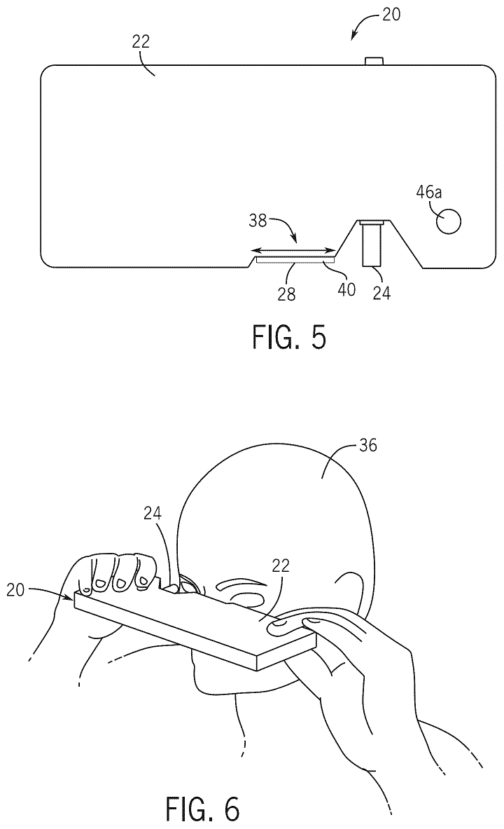

In the embodiments illustrated in FIGS. 5-8, the ophthalmic instrument 20 includes a single anatomical fixation point 28 having a horizontal length 38 to accommodate a range of distances for alignment of the user's eye to the measurement device 24, as adjusted by the user 36.

FIG. 6 shows the ophthalmic instrument 20 in use, with the user 36 holding the body 22, stabilized through the use of two hands, and the anatomical fixation point 28 is rested against a part of the user's anatomy, in this case the user's nose, for alignment and subsequent measurement. The anatomical fixation point 28 may also be designed to rest against the user's cheek, forehead, or brow, or a combination of any of these areas, for example. The anatomical fixation point 28 has a horizontal length 38 to accommodate a range of distances for alignment of the user's eye to the measurement device 24, as adjusted by the user 36. More particularly, while holding the instrument 20 as shown in FIG. 6, with the anatomical fixation point 28 in contact with the user's nose or other anatomy, the user 36 can move the body 22 of the instrument 20 to the left or right along an X-axis until the user's eye aligns with the measurement device 24, which the user 36 can visually detect. For example, an anatomical fixation point 28 having a length 38 of 20 mm could cover pupillary distances (from center of the cornea to nose bridge) of about 55 mm to 75 mm, with a person having a pupillary distance of 55 mm setting the instrument 20 on one end of the anatomical fixation point 28, while a person having a pupillary distance of 75 mm would set the instrument 20 on an opposite end of the anatomical fixation point 28. According to certain embodiments, the anatomical fixation point 28 may have a horizontal length 38 between about 10 mm and about 50 mm, or between about 20 mm and about 40 mm, or between about 25 mm and about 35 mm.

Any apparatus for eye measurement that is stabilized on the nose requires a mechanism to accommodate the distance between the nose fixation point 28 and the center of the user's eye, otherwise known as the monocular pupillary distance (PD). This distance can vary widely among the population. According to certain embodiments, the anatomical fixation point 28 may include a pad 40, such as a nose pad, that is designed to rest the body 22 on the user's nose or other targeted anatomical area. The pad 40 provides comfort and also serves as an alignment indicator, which helps the user 36 properly place the body 22 against the user's nose or other targeted anatomical area. According to certain embodiments, the pad 40 may be a color that is different than the color of the body 22 to enable the user 36 to easily spot the pad 40. Additionally or alternatively, the pad 40 may have a slightly tacky or rough texture or other texture that differs from the texture of the body 22, also designed to enable the user 36 to easily identify the pad 40 for proper placement. The pad 40 may be formed of foam, silicone, or any other medical grade polymer materials, and may be adhered to the anatomical fixation point 28 on the body 22 with adhesive or other suitable attachment means. As the body 22 of the instrument 20 is stabilized with both of the user's hands, the anatomical fixation point 28 requires an appropriate horizontal length 38 of the pad 40 to accommodate a range of monocular pupillary distances (FIG. 5). The horizontal length 38 of the pad 40 may be essentially the same as the horizontal length 38 of the anatomical fixation point 28 in general. This horizontal leeway allows the user 36 to align their eye with the measurement device 24 by sliding the body 22 left or right along the pad 40 to accommodate the measured eye's monocular PD. However, even if alignment of one eye to the measurement device 24 is accomplished, the measurement alone fails to identify which eye is being measured.

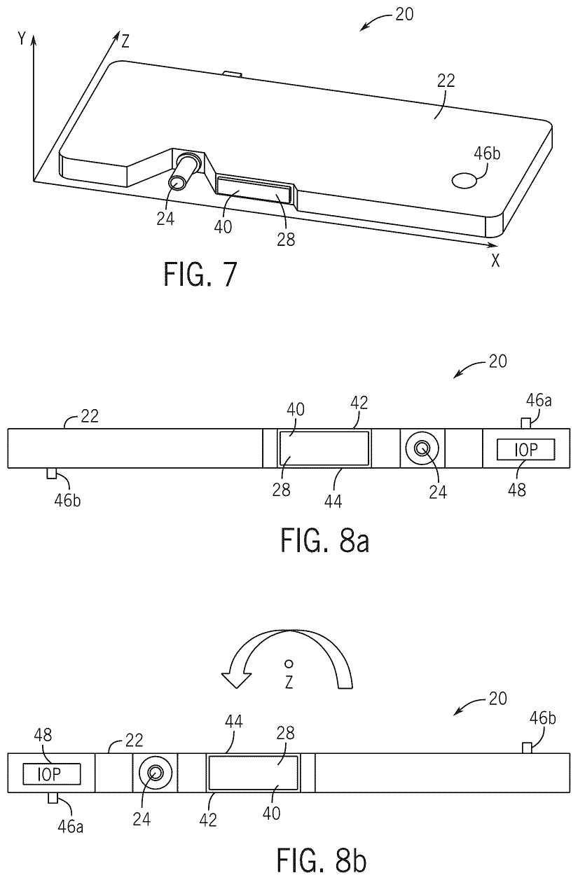

The ophthalmic instrument 20 also includes an eye identification device. According to one or more embodiments, the eye identification device includes a gyroscope or other sensor that detects rotation of the body 22 about a Z-axis 180 degrees when the ophthalmic instrument 20 is inverted or flipped. As the human face is largely symmetric with respect to a Y-axis, eye identification can be realized in combination with the alignment and stability mechanism through the integration of an asymmetry of the body 22 of the instrument 20 with respect to a Y-axis while maintaining a functional symmetry with respect to an X-axis (FIG. 7) and incorporating the gyroscope or other sensor that is housed within the body 22. The embodiment illustrated in FIGS. 1-4 also has asymmetry with respect to a Y-axis, with one of the eye cups 30a housing the measurement device 24 and the other one of the eye cups 30b lacking a measurement device. The eye cup 30b lacking a measurement device may have a non-functional eye-facing surface, which may be an opaque surface, for example. Since the gyroscope or other sensor is housed within the body 22, it is not illustrated in the figures. The term “functional,” as used herein, indicates that while all the features of the body 22 themselves do not have to be exactly symmetric with respect to the X-axis, some symmetric features of the ophthalmic instrument 20, such as the eye cups 30a, 30b or the pad 40 and the measurement device 24, are integral to the functionality due to their symmetry. More particularly, features such as the measurement device 24 as well as the eye cups 30a, 30b or the pad 40 function equally well in both the original position as well as in the inverted or flipped position of the ophthalmic instrument 20.

The asymmetry of the ophthalmic instrument 20 with respect to the Y-axis allows a right eye or a left eye to be measured by rotating the body 22 of the instrument 20 around the Z-axis of the body 22 (FIGS. 8a and 8b) and subsequently sliding the body 22 horizontally to align the eye with the measurement device 24. The ophthalmic instrument 20 maintains the adjusted pupillary distance when the body 22 is flipped, corresponding to the vertical symmetry of the user's face. Also, according to certain embodiments, the functional X-axis symmetry of the ophthalmic instrument 20 allows the user to utilize the same pad 40 in the vertically inverted position to again rest the body 22 after rotating, and again slide the body 22 along horizontally to align to the appropriate monocular pupillary distance of the next eye to be measured, as accommodated by the pad length. As shown in FIG. 8a, a first edge 42 of the pad 40 is on top and a second edge 44 of the pad 40 is on the bottom when the body 22 is ready for alignment with the user's right eye, and when rotated 180 degrees about the Z-axis, as shown in FIG. 8b, the first edge 42 of the pad 40 ends up on the bottom and the second edge 44 of the pad 40 ends up on top when the body 22 is ready for alignment with the user's left eye. The body 22 may include two measurement buttons that allow for right-handed measurement in both the left and right eye orientations of the instrument 20. A first measurement button 46a may be positioned on a top surface of the body 22 on a righthand side of the body 22 when the instrument 20 is in the right eye orientation, and a second measurement button 46b may be positioned on a bottom surface of the body 22 on a lefthand side of the body 22 when the instrument 20 is in the right eye orientation, such that the second measurement button 46b ends up on the top surface of the body 22 on the righthand side of the body 22 when the instrument 20 is rotated 180 degrees about the Z-axis. Incorporation of the gyroscope or other sensor establishes a vertical reference axis of the ophthalmic instrument 20 paired to the eye laterality, wherein rotation of the body 22 around the Z-axis with subsequent inversion of the gyroscope's reference axis indicates which eye is being measured. For example, the gyroscope may be connected to an electronic display panel 48 positioned on the body that indicates the left eye or right eye. The same electronic display panel 48 may also be connected to the measurement device 24 to indicate the pressure data. Alternatively, the pressure data may be indicated on a different electronic display panel 48 than the right or left eye indicator.

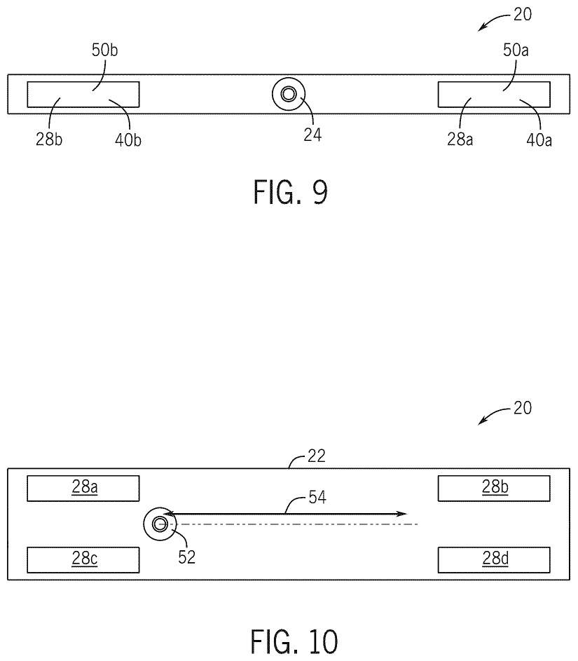

According to another embodiment, illustrated in FIG. 9, the eye identification device includes at least two sensors 50a, 50b each associated with an anatomical fixation point 28a, 28b. As used herein, the term “sensor” refers to a sensor that can detect and respond to a signal or stimulus, such as proximity, contact, movement, rotation, temperature, sound, and the like. The measurement device 24 may be located centrally along the X-axis of the body 22, with a first anatomical fixation point 28a and first sensor 50a positioned on one side of the measurement device 24 and a second anatomical fixation point 28b and second sensor 50b positioned on a second side of the measurement device 24 opposite the first side. A pad 40, such as a nose pad, may also be affixed to each of the anatomical fixation points 28a, 28b. Just like the previously-described embodiments, the body 22 of the instrument 20 is intended to be held with two hands and rested on either the right nose pad 40a or the left nose pad 40b to measure the user's left eye or right eye, respectively. Both pads 40a, 40b are appropriate length in relation to the measurement device 24 to accommodate a wide range of monocular PDs, as adjusted by the user.

A sensor 50a, 50b is placed at or near each anatomical fixation point 28a, 28b to sense when the body 22 of the instrument 20 is rested on the user's anatomy. The sensors 50a, 50b may be contact sensors, proximity sensors, or other suitable sensors. For example, when the body 22 of the instrument 20 is rested on the user's nose against the left nose pad 40b, a contact sensor on or under the left nose pad 40b will detect the contact, which indicates measurement of the user's right eye. Conversely, when the body 22 of the instrument 20 is rested on the user's nose against the right nose pad 40a, a contact sensor on or under the right nose pad 40a will detect the contact, which indicates measurement of the user's left eye. As another example, when the body 22 of the instrument 20 is rested on the user's nose against the left nose pad 40b, a proximity sensor positioned near the left nose pad 40b will detect the presence of the body 22, which indicates measurement of the user's right eye. Conversely, when the body 22 of the instrument 20 is rested on the user's nose against the right nose pad 40a, a proximity sensor positioned near the right nose pad 40a will detect the presence of the body 22, which indicates measurement of the user's left eye. As noted above, the anatomical fixation points 28a, 28b may be designed to rest against the user's nose, cheek, forehead, or brow, or a combination of any of these areas. The sensors 50a, 50b in this embodiment may also be used in combination with the gyroscope embodiment.

The embodiment illustrated in FIG. 10 is similar to the embodiment shown in FIG. 9, but with a slidable measurement device 52. More particularly, the instrument 20 illustrated in FIG. 10 has two anatomical fixation points 28a, 28b designed to rest against a part of the user's anatomy that enable the user to adjust the distance between the anatomical fixation points and a center of the first eye and/or the second eye of the user 36. The embodiment illustrated in FIG. 10 also includes at least one anatomical fixation point 28a positioned on one side of the slidable measurement device 52 and at least one anatomical fixation point 28b positioned on the other side of the slidable measurement device 52, with the slidable measurement device 52 having a horizontal travel distance 54 to accommodate a range of distances for horizontal alignment of the user's first eye and/or second eye to the slidable measurement device 52, as adjusted by the user. For example, the instrument 20 may be in the form of a headset with two of the anatomical fixation points 28a, 28b designed to rest against the user's forehead, and two of the anatomical fixation points 28c, 28d designed to rest against the user's cheeks. Much like the horizontal length 38 of the anatomical fixation point 28 described above, the horizontal travel distance 54 may be between about 40 mm and about 95 mm, or between about 45 mm and about 90 mm, or between about 50 mm and about 85 mm. The eye identification device includes a sensor 50, such that when the slidable measurement device 52 travels horizontally, the sensor 50 senses the slidable measurement device 52 in relation to the user 36 and allows identification of a left eye or a right eye being measured. The sensor 50 may include one or more sensing circuits, for example. The sensing circuits may be configured to sense a location of the slidable measurement device 52 to allow identification of a left eye or a right eye being measured. Alternatively, the sensor 50 may include one or more contact sensors as described above. One or more pads 40 may also be positioned on an outer surface of the body 22 on at least one of the anatomical fixation points 28a, 28b, 28c, 28d. The distance adjusted by the user 36 may represent a pupillary distance. Alternatively, the horizontal travel distance 54 of the slidable measurement device 52 may represent a pupillary distance. The slidable measurement device 52 may be the same type of measurement device 24 described above, such as a rebound tonometer or non-contact tonometer, but is capable of being slid along an X-axis.

The descriptions and figures included herein depict specific implementations to teach those skilled in the art how to make and use the best option. For the purpose of teaching inventive principles, some conventional aspects have been simplified or omitted. Those skilled in the art will appreciate variations from these implementations that fall within the scope of the invention. Those skilled in the art will also appreciate that the features described above can be combined in various ways to form multiple implementations. As a result, the invention is not limited to the specific implementations described above, but only by the claims and their equivalents.

1. An ophthalmic instrument for self-tonometry comprising:
a) a body having a functional symmetry with respect to an X-axis and an asymmetry with respect to a Y-axis, wherein rotation of the body about a Z-axis 180 degrees allows a user to measure a right eye or a left eye; and
b) a measurement device positioned inside or on the body, wherein the measurement device measures intraocular pressure of a first eye and/or a second eye of the user;
c) at least one anatomical fixation point that enables a user to adjust a distance between the at least one anatomical fixation point and a center of the first eye and/or the second eye of the user, wherein the at least one anatomical fixation point rests against a part of the user's anatomy and accommodates a range of distances for alignment of the user's first or second eye to the measurement device, as adjusted by the user; and
d) an eye identification device attached to the body, the eye identification device comprising a sensor that detects rotation of the body about the Z-axis 180 degrees in relation to the Y-axis asymmetry of the body, which allows identification of a left eye or a right eye being measured.

2. The ophthalmic instrument according to claim 1 comprising two of the at least one anatomical fixation points, each of the two anatomical fixation points in the form of eye cups designed to rest against the user's orbit bones.

3. The ophthalmic instrument according to claim 2 wherein the measurement device is positioned within a cavity of a first one of the eye cups and a second one of the eye cups comprises an eye-facing surface that occludes the user's vision.

4. The ophthalmic instrument according to claim 1 wherein the at least one anatomical fixation point rests against at least one of the group consisting of the user's nose, the user's cheek, the user's forehead, and the user's brow.

5. The ophthalmic instrument according to claim 1 further comprising a pad positioned on an outer surface of the body aligned with the at least one anatomical fixation point.

6. The ophthalmic instrument according to claim 1 wherein the at least one anatomical fixation point comprises a horizontal length to accommodate a range of pupillary distances.

7. The ophthalmic instrument according to claim 1 where the distance adjusted by the user represents a pupillary distance.

8. The ophthalmic instrument according to claim 1, wherein the at least one anatomical fixation point comprises a contact sensor configured to sense when the anatomical fixation point of the body is rested against a part of the user's anatomy.

9. The ophthalmic instrument according to claim 1, wherein the sensor is a gyroscope.

10. An ophthalmic instrument for self-tonometry comprising:
a) a body;
b) at least two anatomical fixation points that enable a user to adjust a distance between the anatomical fixation points and a center of the first eye and/or the second eye of the user, wherein the anatomical fixation points rest against a part of the user's anatomy;
c) a measurement device positioned inside or on the body, wherein the measurement device measures intraocular pressure of a first eye and/or a second eye of a user, with a first anatomical fixation point positioned on a first side of the measurement device and a second anatomical fixation point positioned on a second side of the measurement device; and
d) an eye identification device attached to the body, the eye identification device comprising a sensor, such that when the body is fixated to the user via one of the anatomical fixation points the sensor senses the user in relation to the measurement device and allows identification of a left eye or a right eye being measured.

11. The ophthalmic instrument according to claim 10 wherein the at least two anatomical fixation points comprise two eye cups designed to rest against the user's orbit bones.

12. The ophthalmic instrument according to claim 11 wherein the measurement device is positioned within a cavity of a first one of the eye cups and a second one of the eye cups comprises an eye-facing surface that occludes the user's vision.

13. The ophthalmic instrument according to claim 10 wherein the at least two anatomical fixation points each rest against at least one of the group consisting of the user's nose, the user's cheek, the user's forehead, and the user's brow.

14. The ophthalmic instrument according to claim 10 wherein each of the anatomical fixation points has a length to accommodate a range of distances for horizontal alignment of the user's first eye and/or second eye to the measurement device, as adjusted by the user

15. The ophthalmic instrument according to claim 10 wherein the sensor comprises one or more contact sensors.

16. The ophthalmic instrument according to claim 15 wherein the one or more contact sensors is configured to sense fixation upon at least one of the anatomical fixation points and allow identification of a left eye or a right eye being measured.

17. The ophthalmic instrument according to claim 10 wherein the sensor comprises one or more proximity sensors.

18. The ophthalmic instrument according to claim 17 wherein the one or more proximity sensors is configured to sense a left eye or a right eye being measured.

19. The ophthalmic instrument according to claim 10 further comprising a pad positioned on an outer surface of the body to align with at least one of the anatomical fixation points.

20. An ophthalmic instrument for self-tonometry comprising:
a) a body;
b) at least two anatomical fixation points that enable a user to adjust a distance between the anatomical fixation points and a center of the first eye and/or the second eye of the user, wherein the anatomical fixation points rest against a part of the user's anatomy;
c) a measurement device positioned inside or on the body, wherein the measurement device measures intraocular pressure of a first eye and/or a second eye of a user, with at least one anatomical fixation point positioned on a first side of the measurement device and at least one anatomical fixation point positioned on a second side of the measurement device, with the measurement device having a horizontal travel distance to accommodate a range of distances for horizontal alignment of the user's first eye and/or second eye to the measurement device, as adjusted by the user; and
d) an eye identification device attached to the body, the eye identification device comprising a sensor, such that when the measurement device travels horizontally the sensor senses the measurement device in relation to the user and allows identification of a left eye or a right eye being measured.

21. The ophthalmic instrument according to claim 20 wherein one of the anatomical fixation points rests against at least one of the group consisting of the user's nose, the user's forehead, the user's cheek, and the user's brow.

22. The ophthalmic instrument according to claim 20 wherein the sensor comprises one or more sensing circuits configured to sense measurement device location to allow identification of a left eye or a right eye being measured.

23. The ophthalmic instrument according to claim 20 wherein the sensor comprises one or more contact sensors.

24. The ophthalmic instrument according to claim 23 wherein the one or more contact sensors is configured to sense contact at a location to identify a left eye or a right eye being measured.

25. The ophthalmic instrument according to claim 20 further comprising a pad positioned on an outer surface of the body on at least one of the anatomical fixation points.

26. The ophthalmic instrument according to claim 20 wherein the distance adjusted by the user represents a pupillary distance.

27. The ophthalmic instrument according to claim 20 wherein the travel distance of the measurement device represents a pupillary distance.
